(19) United States
(12) Patent Application Publication
(10) Pub. No.: US 2003/0129680 A1
(43) Pub. Date: Jul. 10, 2003

(54) Multi-analyte assay device

(76) Inventor: Thomas Patrick O'Connor JR., Westbrook, ME (US)

Correspondence Address: Lisa M.W. Hillman, Ph.D, McDonnell Boehnen Hulbert & Berghoff, 32nd Floor, 300 S. Wacker Drive, Chicago, IL, 60606, US

(21) Appl. No.: 10/280,884

(22) Filed: Oct. 25, 2002

Related U.S. Application Data
(60) Provisional application No. 60/335,367, filed on Oct. 31, 2001.

(51) Int. Cl. G01N 33/554; G01N 33/569; C12M 1/34

(52) U.S. Cl. 435/7.32; 435/287.2

(57) ABSTRACT

The invention provides devices and methods for detecting the presence or absence of Dirofilaria immitis, Borrelia burgdorferi, and Ehrlichia canis in a sample.

### PRIORITY INFORMATION

This application claims the benefit of U.S. Provisional Application Serial No. 60/335,367 filed Oct. 31, 2001, which is incorporated by reference herein in its entirety.

### TECHNICAL AREA OF THE INVENTION

The invention provides devices and methods for detecting the presence or absence of Dirofilaria immitis, Borrelia burgdorferi, and Ehrlichia canis in a sample.

### BACKGROUND OF THE INVENTION

Heartworm disease is caused by the filarial nematode D. immitis and has worldwide distribution. The insect vector for D. immitis is the mosquito. Adult worms inhabit the blood and vascular tissue of mammals, including, for example, dogs, especially in the heart and adjacent blood vessels. D. immitis often interferes with heart functions and blood circulation and can damage other vital organs. The detection of heartworm antigen is diagnostic for infection by D. immitis.

Ehrlichiosis is a tick-borne disease of mammals, including dogs, caused by the rickettsial parasite E. canis. Replication of the organism occurs within infected mononuclear cells and spreads to organs containing mononuclear phagocytes. Infection can result in thrombocytopenia, leukopenia and/or anemia. Clinical signs of infection include fever, dyspnea, weight loss, hemorrhages and epistaxis. Diagnosis of canine ehrlichiosis has been made by observation of typical clinical signs and by the measurement of a significant antibody titer to E. canis.

Lyme disease is caused by the spirochete Borrelia burgdorferi. B. burgdorferi is transmitted to mammals through the bite of an infected tick. Symptoms can include fever, arthritis, shifting leg lameness, articular swelling, large lymph nodes, anorexia, and general malaise between 2 and 5 months after exposure to the tick. Untreated animals can develop chronic progressive arthritis.

Methods and devices are needed in the art for the detection of D. immitis, B. burgdorferi, and E. canis analytes in a sample.

### SUMMARY OF THE INVENTION

It is an object of the invention to provide reagents and methods for detecting the presence or absence of D. immitis, B. burgdorferi, and E. canis specific antigens or antibodies in a sample. This and other objects of the invention are provided by one or more of the embodiments described below.

One embodiment of the invention provides a device for detection of Dirofilaria immitis, Borrelia burgdorferi, and Ehrlichia canis antigens, antibodies, or fragments thereof. The device comprises an antibody that specifically binds a D. immitis antigen immobilized on a solid support at a distinct location; a polypeptide that specifically binds an antibody specific for B. burgdorferi immobilized on the solid support at a distinct location; and a polypeptide that specifically binds an antibody specific for E. canis immobilized on the solid support at a distinct location.

Another embodiment of the invention comprises a device for the detection of Dirofilaria immitis, Borrelia burgdorferi, and Ehrlichia canis antigens, antibodies, or fragments thereof. The device comprises an elongated solid phase flow matrix comprising a first region for the receipt of a fluid sample; a second region wherein an antibody that specifically binds a D. immitis antigen is immobilized at a distinct location; a polypeptide that specifically binds an antibody specific for B. burgdorferi is immobilized at a distinct location, and a polypeptide that specifically binds an antibody specific for E. canis is immobilized at a distinct location; and a third region for application of a liquid detector reagent capable of removing unbound substances from the second region. The second region is positioned intermediate to the first region and the third region. The device further comprises an absorbent reservoir of high volume capacity, wherein prior to use of the device, the absorbent reservoir is not in fluidic contact with the flow matrix; the device further comprising means for establishing fluidic contact between the absorbent reservoir and the flow matrix at positions selected so that the second region is between the absorbent reservoir and the third region. The device also comprises a sealed container of the liquid detector reagent positioned to be introduced at the third region of the matrix. The flow matrix and the regions thereof are sized and positioned to cause the fluid sample to flow initially along the elongated flow matrix in one direction toward and through the second region, and subsequently, upon introduction of the liquid detector reagent into the third region of the flow matrix, the liquid detector reagent to flow along the elongated flow matrix in a second direction opposite the first direction, through the second region, and into the absorbent reservoir, drawing unbound substances with it.

Still another embodiment of the invention provides a method for performing an assay that determines presence or absence of Dirofilaria immitis antigens, Borrelia burgdorferi antibodies, and Ehrlichia canis antibodies in a fluid sample by detecting binding of the antigens and antibodies to at least one immobilized antibody that specifically binds a D. immitis antigen, at least one immobilized polypeptide that specifically binds an antibody specific for B. burgdorferi, and at least one immobilized polypeptide that specifically binds an antibody specific for E. canis after washing unbound material from the immobilized antibody and polypeptides. The method comprises providing an elongated solid phase flow matrix, the solid phase flow matrix capable of driving capillary fluid movement, the flow matrix further comprising a first region for the receipt of a fluid sample; a second region at which the at least one antibody and at least one polypeptides are immobilized; and a third region for application of a liquid reagent capable of removing unbound substances from the second region; the second region being positioned intermediate to the first region and the third region. Also provided is an absorbent reservoir of high volume capacity, wherein, prior to performing the method, the absorbent reservoir is not in fluidic contact with the flow matrix. The fluid sample is applied to the first region of the flow matrix and allowed to flow in a first direction through the second region. A liquid detector reagent is introduced into the flow matrix at the third region. The absorbent reservoir is moved into fluidic contact with the flow matrix, such that the sample and the liquid detector regent flow in a second direction, opposite to the first direction. D. immitis antigens, B. burgdorferi antibodies, and E. canis antibodies bound at the second region are detected.

Even another embodiment of the invention provides a device for performing an assay that determines presence or absence of Dirofilaria immitis antigens, Borrelia burgdorferi antibodies, and Ehrlichia canis antibodies in a fluid sample by detecting binding of the antigens and antibodies to at least one immobilized antibody that specifically binds a D. immitis antigen, at least one immobilized polypeptide that specifically binds an antibody specific for B. burgdorferi, and at least one immobilized polypeptide that specifically binds an antibody specific for E. canis by detecting binding of the D. immitis antigens, B. burgdorferi antibodies, and E. canis antibodies to at least one immobilized antibody or polypeptide after washing unbound material from the immobilized antibody and polypeptides. The device comprises an elongated fluid flow matrix comprising a first segment for receiving a fluid sample, a second region at which the antibody and polypeptides are immobilized, and a third region for application of a liquid detector reagent capable of removing unbound substances from the second region. The device further comprises an absorbent reservoir of high volume capacity, a sealed container of the liquid detector reagent positioned to be introduced at the third region of the flow matrix, and, optionally, at least one soluble barrier positioned to block flow of the liquid detector reagent from the container to the absorbent reservoir, the second region being positioned intermediate to the third region and the absorbent reservoir. The soluble barrier blocks the flow of the liquid detector reagent to the absorbent reservoir until after the sample has flowed from the first region through the second region, at which point the barrier dissolves permitting the liquid detector reagent to flow.

Methods and devices of the invention provide accurate and efficient detection of the presence or absence of three different mammalian pathogens with little or no cross reaction between each specific detection reaction. The methods and devices of the present invention provide a number of advantages in detecting analytes associated with D. immitis, E. canis and B. burgdorferi infection. For example, devices and methods of the invention facilitate unusually sensitive analyte detection. Sample liquid is flowed within devices of the invention in such a manner that analyte is in contact with mobile assay reagents (e.g., an enzyme-labeled antibody) for a substantial portion of the assay, and the opportunity for analyte contact with immobilized analyte capture reagents is present both from forward flow and from reverse flow. Maximizing analyte contact with assay reagents maximizes the efficiency of analyte capture, facilitating an analytical method that requires only a small volume of test sample and that provides for unusually sensitive detection of even scant quantities of analyte.

Moreover, reversible flow provides a semi-automated format whereby detector reagent can enter the reactive zone following removal of unbound sample and unbound labeled specific binding reagents (e.g., enzyme-antibody conjugate) by wash reagent. This minimizes contact between the detector reagent (e.g., substrate) and unbound labeled specific binding reagents, reducing background (e.g., background color reaction) and, thereby, increasing sensitivity. In addition, the semi-automated format facilitates case of performance by reducing operator involvement.

In summary, reversible flow techniques of the instant invention facilitate assays that are of low background and high specificity. In addition, the automated nature of the immuno-chromatographic process significantly reduces the level of technical sophistication required of an individual performing assays described herein, facilitating assays that can be carried out in an environment remote from a laboratory and by reasonably untrained practitioners.

### BRIEF DESCRIPTION OF THE DRAWINGS

FIG. 1 is a top plan view of a device for carrying out a reversible flow chromatographic binding assay of the present invention.

FIG. 2A is a cross-sectional illustration of the device of FIG. 1, showing the position of the top portion after operator activation.

FIG. 2B is a cross-sectional illustration of the device of FIG. 1, showing the position of the top portion before operator activation.

### DETAILED DESCRIPTION OF THE INVENTION

Devices of the Invention

A structure of an exemplary device of the invention is described in U.S. Pat. No. 5,726,010, which is herein incorporated by reference in its entirety. Devices of the invention can make use of bi-directional capillary flow (i.e., reversible flow) to transport an analyte-containing sample first in one direction and then in the opposite direction along an elongated capillary flow matrix. Such reversible flow makes more efficient use of available sample by maximizing analyte contact with specific binding reagents (i.e., both during forward flow and during reverse flow). Reversible flow also facilitates elimination of unreacted sample and unbound reagents from the detection zone; a detector/wash reagent is flowed along the assay device in the opposite direction to the original sample flow drawing with it unbound or unreacted constituents. This increases the sensitivity of the assay by removing reagents which contribute to non-specific background.

In general, a first aspect of the invention features a device for performing an assay that determines the presence or absence of an analyte (e.g., an antigen derived from D. immitis, B. burgdorferi, or E. canis or an antibody or fragment thereof specific for D. immitis, B. burgdorferi or E. canis) in a fluid sample by detecting binding of the analyte to at least one immobilized analyte capture reagent, e.g. an antibody or fragment thereof specific for D. immitis, B. burgdorferi or E. canis or a polypeptide specific for D. immitis, B. burgdorferi or E. canis). To facilitate detection, unbound material is washed from the immobilized analyte capture reagent zone. The device involves an elongated solid phase flow matrix that is capable of driving capillary fluid movement and means to detect analyte bound at the second region. The flow matrix itself includes the following regions: (i) a first region adapted for receipt of the fluid sample, (ii) a second region at which the analyte capture reagent is immobilized, (iii) a third region for application of a liquid reagent capable of removing unbound substances from the second region; and (iv) an absorbent reservoir that has a high volume of absorbent capacity. The second region is positioned intermediate to the first region and the third region and intermediate to the absorbent reservoir and the third region. The flow matrix and the regions thereof are sized and positioned to cause the fluid sample to flow initially along the elongated flow matrix in one direction toward and through the second region, and subsequently, to cause the liquid reagent to flow along the elongated flow matrix in a second direction opposite the first direction, through the second region, and into the absorbent reservoir, drawing unbound substances with it.

One specific form of the assay method described below is a sandwich format in which sample analyte is contacted with non-immobilized labeled specific binding reagents (e.g., an enzyme-antibody conjugate). The analyte is immobilized (at a detection zone) as a result of its binding to an analyte capture reagent (e.g., analyte-specific antibody or polypeptide bound to a solid substrate, e.g., Latex beads or the assay device itself). Complex formation (e.g., antibody-antigen immunocomplexes), at the detection zone is assayed either directly (e.g., when using a radioactive, fluorescent, or light-absorbing label) or following reaction with a detector reagent (e.g., a chromogenic substrate that reacts with the enzyme component of an enzyme-antibody conjugate).

Generally, a binding assay using the methods and devices of the instant invention is performed as follows. A sample containing an analyte is applied to a device of the invention via a sample application means and allowed to flow along, and eventually to saturate, a flow matrix. This facilitates sequential complex formation; an analyte binds first to a non-immobilized labeled specific binding reagent and then to an immobilized analyte capture reagent. The absorbent reservoir is contacted with a saturated flow matrix (e.g., mechanically or by dissolution of an optional soluble film that serves to separate the absorbent reservoir from the flow matrix), thereby reversing the fluid flow. Finally, detector and/or wash solution is delivered to the flow matrix (e.g., by piercing a storage vessel containing the solution(s) or by allowing the sample to dissolve a soluble film that serves to separate the liquid reagents from the flow matrix). Liquid reagents remove unbound sample molecules and unbound labeled specific binding reagent and also facilitate detection of analyte complexes (at the location of the immobilized analyte capture reagent). An analyte complex comprises an immobilized analyte capture reagent specifically bound to an analyte molecule. Contact of the flow matrix with the absorbent reservoir and delivery of liquid reagents is preferably performed simultaneously.

The overall sequencing of the above steps is controlled by the flow of liquid within the flow matrix and the physical positioning of the sample and liquid reagent entry points relative to the position of the deposited labeled specific binding reagents and analyte capture reagents. Operator involvement is, in general, limited to a maximum of three steps: application of the sample, one-step release of stored liquid reagents (i.e., substrate/wash solution), and mechanical contacting of the absorbent reservoir with the flow matrix. Use of dissolvable films to control absorbent reservoir contact with the flow matrix and/or release of the detector/wash solution(s) reduces operator involvement to two steps or even a single step. Additionally, the use of a direct visualization label, such as a latex particle, gold sol or dye sol can be used to reduce operator involvement.

To facilitate a reversible flow-type binding assay, a device according to the invention generally comprises the following components: a sample entry means; a flow matrix that is capable of supporting capillary liquid flow and that initially directs flow in the forward direction (i.e., away from the sample entry means); an absorbent reservoir positioned adjacent to the sample entry means that can be fluidically coupled to the flow matrix in order to promote liquid flow in the reverse direction (i.e., back toward the sample entry means); and a liquid reagent entry means located at the opposite end of the device that facilitates delivery of a detector reagent and/or a wash reagent upon reversal of the liquid flow.

There now follow descriptions of particular test devices according to the invention. These examples are provided for the purpose of illustrating, not limiting, the invention.

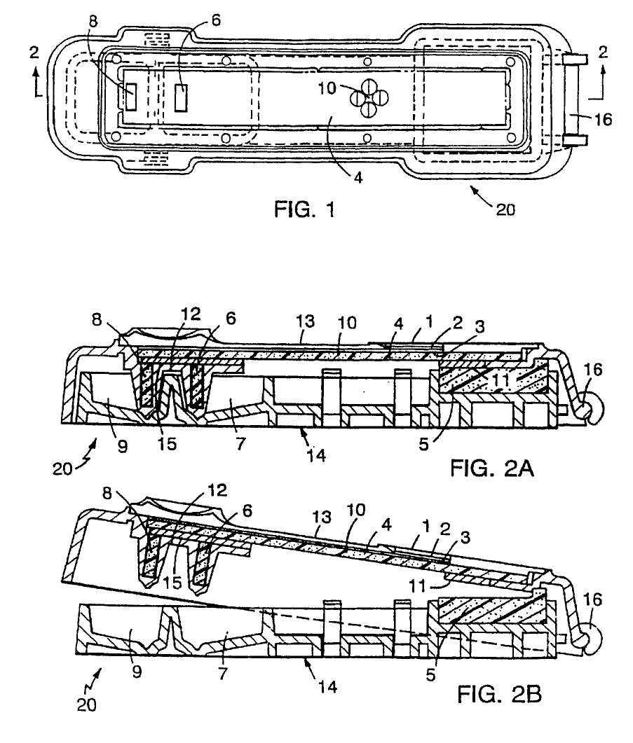

FIGS. 1 and 2 depict one example of a device 20 according to the invention. Components of the device are enclosed within an upper housing portion 13 and a lower housing portion 14, pivotably disposed with respect to each other by means of a hinge 16. Such a housing serves to properly hold the components in place and to allow delivery of a sample to the internal flow matrix as well as to allow an operator to visually monitor assay results. The pivotal connection initially holds the two portions of the housing apart (allowing “forward” flow). Operator activation is accomplished by squeezing components 13 and 14 together, contacting the flow matrix with the absorbent reservoir and releasing the liquid reagents (as described below), enabling “reverse flow.” The absorbent reservoir can be an absorbant pad that is capable of accommodating a volume of liquid in excess of the total volume sample and the total volume of all added liquid reagents (e.g., detector reagent or wash reagent).

To carry out a binding assay using such a device, a fluid sample is applied through a sample entry cup 1. The fluid sample is drawn into the flow matrix 4 as follows. A sample can flow through a sample prefilter pad 2, that removes interfering particulate matter and, through a labeled specific binding reagent pad 3 upon that labeled specific binding reagent has been deposited and dried. Contact of the labeled specific binding reagent pad with the fluid sample results in dissolution of the labeled specific binding reagent into the sample, allowing sample analyte to bind to the labeled specific binding reagent; positioning of the labeled specific binding reagent pad adjacent to the sample entry cup increases the quantity of sample that contacts the dried reagent. Sample and labeled specific binding reagent are then drawn, by capillary action, into the flow matrix 4 and transported in the “forward” direction within the physical structure of the matrix towards and past the reactive zone 10 where immobilized analyte capture reagent has been incorporated into the flow matrix. At the reactive zone 10, all binding species are present (i.e., sample, labeled specific binding reagent and immobilized analyte capture reagent). Fluid flow continues in the forward direction until the flow matrix 4 is saturated, at which point, fluid flow ceases. At this time, housing components 13 and 14 are squeezed together by the operator (as described above), bringing the flow matrix 4 into contact with the absorbent reservoir 5. The absorbent reservoir is positioned toward one end of matrix 4 so as to draw the fluid out of the matrix and to reverse the direction of fluid flow within the device.

Upon flow reversal, liquid reagents are delivered to the flow matrix. In the device illustrated in FIGS. 1 and 2, such liquid reagents include a wash reagent and a detector reagent. The wash reagent is stored in a wash reagent storage vessel 7 and is delivered, by the wash reagent delivery wick 6 into the flow matrix 4. The purpose of the wash reagent is to transport unbound sample and unbound labeled specific binding reagent along the flow matrix 4 and away from the reactive zone 10. Detector reagent is stored in the detector reagent storage vessel 9 and is delivered, by the detector reagent delivery wick 8 into the flow matrix 4. The detector reagent facilitates analyte detection. The device depicted in FIGS. 1 and 2 illustrates a physical linkage of the delivery wicks within the lance 12 which serves to both pierce the storage vessels and deliver the reagent to the flow matrix. A lance can be a component that is capable of piercing a seal of a liquid reagent container. A wick can facilitate flow of the liquid reagents out of their storage container and into the flow matrix.

In another embodiment of the invention one or more labeled specific binding reagents can be mixed with a test sample prior to application to a device of the invention. In this case it is not necessary to have labeled specific binding reagents deposited and dried on a specific binding reagent pad. A labeled specific binding reagent, whether added to a test sample or pre-deposited on the device, can be for example, a labeled antibody specific for D. immitis. For example, a D. immitis-specific antibody raised in a chicken conjugated with horseradish peroxidase can be used as a labeled specific binding reagent. Other examples of labeled specific binding reagents include a polypeptide specific for a B. burgdorferi or E. canis antibody, such as SEQ ID NOs: 1-4, conjugated to horseradish peroxidase. The labeled specific binding reagent can be in a solution, such as buffered protein serum. A labeled specific binding reagent can also be, for example, an antibody specific for D. immitis or polypeptides specific for B. burgdorferi or E. canis antibodies conjugated to a latex particle, gold sol or dye sol.

A liquid reagent is a fluid that transports unbound material (e.g., unreacted fluid sample and unbound specific binding reagents) away from the second region. A liquid reagent can be a wash reagent and serve only to remove unbound material from the second region, or it can include a detector reagent and serve to both remove unbound material from the second region and to facilitate analyte detection. Two or more liquid reagents can be present in a device, for example, a device can comprise a liquid reagent that acts as a wash reagent and a liquid reagent that acts as a detector reagent and facilitates analyte detection. Where both types of liquid reagents are present at the third region of a flow matrix, the liquid reagent that acts as a wash reagent is closer to the immobilized analyte capture reagent zone than the liquid detector reagent is to the analyte capture reagent zone.

A liquid reagent can further include a limited quantity of an “inhibitor”, i.e., a substance that blocks the development of the detectable end product. A limited quantity is an amount of inhibitor sufficient to block end product development until most or all excess, unbound material is transported away from the second region, at which time detectable end product is produced.

The linkage of the delivery wicks facilitates the release of the two stored liquid reagents with a single action. Sequential utilization of the two reagents, i.e., wash reagent followed by detector reagent is accomplished by delivering the wash reagent closer to the absorbent reservoir 5 than the detector reagent. Fluid flow toward the absorbent reservoir causes the wash reagent to be pulled into the flow matrix 4 by capillary force. Once the volume of the delivered reagent has been absorbed into the flow matrix, displacing unbound sample and unbound labeled specific binding reagent, detector reagent is delivered into the flow matrix 4 by capillary force. Detector reagent displaces the wash reagent in the direction of the absorbent reservoir 5. When the detector reagent flows into the reactive zone 10, complex formation is detectable, and the assay procedure is complete.

In another embodiment of the invention, a detector reagent can act both to remove unbound sample and reagents from the reactive zone and to facilitate analyte detection. Such a device can be designed essentially as shown in FIGS. 1 and 2, except that the device includes a single reagent storage vessel and a single reagent delivery wick (e.g., included as a component of the lance). As described above, sample is added to the device and, at some point after addition (and preferably, after sample has saturated the flow matrix), the device is operator activated (as described above). The detector reagent storage vessel is pierced by the lance (containing a delivery wick) and the detector reagent delivered to the flow matrix. Reversal of the fluid flow (also as described above) draws the detector reagent into the flow matrix by capillary force. As the detector reagent flows towards the absorbent reservoir, it displaces the fluid in the flow matrix, clearing the matrix, and importantly, clearing the reactive zone of unbound sample and unbound labeled specific binding reagent.

In the case of a labeled specific binding reagent conjugated to a radioactive, fluorescent, or light-absorbing molecule, the detector reagent acts merely as a wash solution facilitating detection of complex formation at the reactive zone by washing away unbound labeled reagent.

In the case of a specific binding reagent conjugated, e.g., to an enzyme, the detector reagent includes, e.g., a substrate that produces a detectable signal upon reaction with the enzyme-antibody conjugate at the reactive zone. In such a case, a finite quantity of inhibitor reagent can be incorporated into an inhibitor reagent pad located at the junction of the detector reagent dispense cup and the flow matrix or can be dried directly on to the flow matrix between the detector reagent dispense cup and the reactive zone. When the finite quantity of inhibitor migrates out of the reactive zone, detector reagent produces a detectable signal upon contact with the labeled specific binding reagent.

To ensure proper operation, any of the devices described herein can further include various binding reagents immobilized at the reactive zone 10 at locations distinct from the analyte capture reagent(s). For example, an immunoreagent that recognizes a species-specific (e.g., canine specific) antibody portion of a labeled specific binding reagent or an enzyme portion of an enzyme-labeled reagent can be included as a positive control to assess the viability of the reagents within the device. For example, a positive control can comprise an anti-horseradish peroxidase antibody that has been raised in, for example, a goat or a mouse. Additionally, a reagent, e.g., an antibody isolated from a non-immune member of the species from which the antibody portion of the enzyme-antibody conjugate was derived can be included as a negative control to assess the specificity of immunocomplex formation.

To maximize automation, a device of the invention can further optionally include a soluble film 11 which separates the flow matrix 4 from the absorbent reservoir 5. For example, a soluble film can be located at the base of the sample entry port which first directs flow of the sample liquid toward the specific binding reagents; the dissolution of the film (by residual sample in the sample entry cup) then reverses the direction of the capillary flow through the device by allowing contact between an absorbent reservoir (located beneath the film) and the flow matrix. The timed dissolution of this film increases the period available for immunocomplex formation, without requiring precisely-timed addition(s) of one or more reagents by the operator. A second optional soluble film can be located at the base of the detector/wash dispenser cup(s). Dissolution of this film by sample that has traversed the length of the flow matrix allows contact of the detector/wash with the flow matrix and, upon reversal of the fluid flow and emptying of the flow matrix at the detector/wash entry point, the detector/wash is flowed by capillary action in the direction of the immobilized binding reagents. Sample added to the flow matrix at the sample entry port 1 is thereby flowed in a single direction (i.e., away from the absorbent reservoir) maximizing the amount of sample that flows past the reactive zone 10. The film is dissolved slowly by the fluid sample and, upon dissolution, contact occurs between the absorbent pad 5 and the flow matrix 4 and promotes a reversal of the fluid flow. An optional soluble film 15 can also be positioned between the liquid reagent storage vessels 6 and 8 and the flow matrix. Dissolution of the film by fluid that has flowed to the end of the matrix (i.e., the end distal to the sample entry port 1) allows delivery of the liquid reagents to the flow matrix. Reverse fluid flow draws the reagents into the matrix by capillary force.

The fundamental components of the invention can be packaged as a single unit or housed as several units for multiple-sample devices. Various packaging options in which liquid reagent storage reservoirs or sample entry points are shared between several flow matrix components can also be envisioned. In one particular example, the device contains multiple regions within the reactive zone, each including a different analyte capture reagent (e.g., one can include an immobilized antibody that specifically binds a D. immitis antigen, an immobilized polypeptide that specifically binds an antibody specific for B. burgdorferi, and an immobilized polypeptide that specifically binds an antibody specific for E. canis); a single biological sample (e.g., a sample of canine serum) is assayed for the presence of one or more of these microbes.

In one embodiment of the invention, the reactive zone 10 can be seen from the outside of the housing, allowing ready detection of assay results. The sample entry cup 1 can be designed such that the volume of the cup is at least as large as the total volume of sample required to perform the assay. In addition, an absorbent pad 5 can be of sufficient size to accommodate the total volume of sample as well as all added liquid reagents (i.e., detector reagent and wash reagent).

A flow matrix material can possess the following characteristics: (1) low non-specific affinity for sample materials and labeled specific binding reagents, (2) ability to transport a liquid by capillary action over a distance with a consistent liquid flow across the matrix, and (3) ready binding to immobilized specific binding reagents, (e.g., by covalent or non-covalent attachment or by physical entrapment). Materials possessing these characteristics include fibrous mats composed of synthetic or natural fibers (e.g., glass or cellulose-based materials or thermoplastic polymers, such as, polyethylene, polypropylene, or polyester); sintered structures composed of particulate materials (e.g., glass or various thermoplastic polymers); or cast membrane films composed of nitrocellulose, nylon, polysulfone or the like (generally synthetic in nature). The invention can utilize a flow matrix composed of sintered, fine particles of polyethylene, commonly known as porous polyethylene; such materials can possess a density of between 0.35 and 0.55 grams per cubic centimeter, a pore size of between 5 and 40 microns, and a void volume of between 40 and 60 percent. Particulate polyethylene composed of cross-linked or ultra high molecular weight polyethylene can be used. A flow matrix composed of porous polyethylene possesses all of the features listed above, and in addition, is easily fabricated into various sizes and shapes. In one embodiment of the invention, 20-30 micron porous polyethylene is used.

Materials suitable for use as an absorbent reservoir are highly absorbent, provide capacity in excess of the volume of the fluid sample plus the added liquid reagents, and are capable of absorbing liquids from the flow matrix by physical contact as the sole means of fluid transfer between the two materials. A variety of materials and structures are consistent with these requirements. Fibrous structures of natural and synthetic fibers such as cellulose and derivitized cellulose (e.g., cellulose acetate) can be used. The fibers of the material can be oriented along a particular axis (i.e., aligned), or they can be random. One embodiment of the invention utilizes non-aligned cellulose acetate fibers of density range 0.1 to 0.3 grams per cubic centimeter and void volume of 60 to 95 percent.

Materials suitable for use as a labeled reagent deposit pad can possess the following properties: (1) high liquid void volume, facilitating an even exposure of the fluid sample to the solid material upon which the labeled binding reagent has been dried, (2) a rapid flow property such that the rate of sample entry into the flow matrix is not governed by the labeled reagent pad, (3) material surface properties that do not adversely affect the efficacy of the deposited specific binding reagents and that allow ready reconstitution of the dried reagents, and (4) ability to establish liquid flow between the absorbent pad and the flow matrix (e.g., compressibility without loss of flow characteristics). In general, materials having the above properties are fibrous structures with low density fiber configurations. Materials composed of synthetic fibers, such as polyester have the advantage of inert surfaces and low density structures. In an alternative embodiment of the invention, a labeled reagent deposit pad is composed of a random alignment of polyester fibers that are heat-needled into a mat structure with a material density of 2 to 12 ounces of polyester per square yard.

The housing can be watertight to prevent leakage and can be manufactured from an inert material, such as polymer materials, which are easy to fabricate.

Materials suitable for use as a dissolvable film are dissolved by the fluid sample, do not interfere with specific binding or chemical reactions necessary to the assay, and do not adversely affect the flow properties of the liquids within the flow matrix. In general, materials having the above properties are polymers of molecular weight 3,000 to 10,000,000, including polyvinyl alcohol, polyethylene oxide, and methyl cellulose. In one embodiment of the invention, the film is polyvinyl alcohol of thickness 0.0016 inches;

The signal producing system can generally involve the production of a detectable signal, for example, due to a radioactive, fluorescent, or light-absorbing molecule. Such a molecule preferably does not interfere with the ability of the labeled specific binding reagent to traverse the flow matrix. In addition, if the detectable end product is produced upon reaction with detector reagent, it is preferable that end product precipitate out of solution resulting in a localized signal rather than a “lateral streak” that extends throughout the flow matrix. Such a signal producing system can involve an enzyme and a substrate. One example of a substrate that forms an insoluble end product following reaction with the enzyme, alkaline phosphatase, is indoxyl phosphate. An example of a substrate that produces an insoluble end product following reaction with the enzyme, horseradish peroxidase, is tetramethylbenzidine.

Alternatively, the signal producing system can comprise an enzyme or coenzyme that produces an end-product that absorbs light (e.g., a dye) or that emits light upon irradiation or chemical reaction, i.e., a fluorescent or chemiluminescent molecule, respectively. A large number of enzymes and coenzymes for providing such products are indicated in U.S. Pat. No. 4,275,149 and U.S. Pat. No. 4,318,980 (hereby incorporated by reference). The product of the enzyme reaction will usually be a dye or fluorescer. A large number of illustrative fluorescers are also indicated in U.S. Pat. No. 4,275,149, that is incorporated by reference.

Of particular interest is the enzyme horseradish peroxidase that produces a colored product when reacted with the substrate, 4-chloro-1-napthol. One commercially-available substrate solution is termed TM Blue and is available from TSI Incorporated (Worcester, Mass.). Also of interest are enzymes that involve the production of hydrogen peroxide and the use of the hydrogen peroxide to oxidize a dye precursor to a dye. Particular combinations include saccharide oxidases e.g., glucose and galactose oxidase, or heterocyclic oxidases, such as uricase and xanthine oxidase, coupled with an enzyme that employs the hydrogen peroxide to oxidize a dye precursor, e.g., peroxidase, microperoxidase, and cytochrome C oxidase. Additional enzyme combinations can be found in the subject matter incorporated by reference.

The detector reagent can also serve to remove unbound sample and binding reagents from the flow matrix by inclusion in the detector solution of a limited quantity of inhibitor; such an inhibitor blocks the development of a visible end product. In general, a suitable inhibitor must dissolve quickly and completely into the detector reagent solution. The inhibitor blocks end product development, e.g., by reversibly inhibiting the activity of the enzyme conjugate, by chemically consuming substrate molecules, or by acting as an alternative substrate that produces no visible end product upon reaction with the enzyme.

In particular examples, the enzyme alkaline phosphatase is inhibited by a 0.05M sodium phosphate solution at pH 6 to pH 7; inhibition is due to decreased enzyme activity (resulting from a solution pH that is lower than alkaline phosphatase's optimum pH of 10). In another example the enzyme horseradish peroxidase is inhibited by 0.025M sodium metabisulfite. In this case, end product formation is blocked because the inhibitor chemically consumes the electron-donating peroxide substrate (i.e., by reducing available substrate). Horseradish peroxidase can also be inhibited by 0.05M ascorbic acid. Ascorbic acid serves as an alternative horseradish peroxidase substrate, reacting with the enzyme, but producing no visible end product.

The quantity of added inhibitor is determined empirically. A suitable amount of inhibitor blocks production of end product until most or all of the unbound labeled binding reagent is removed from the reactive zone, at which time, detectable end product is produced.

Methods and devices of the invention facilitate sandwich or competition-type specific binding assays. In the case of a sandwich assay, analyte capture reagents are immobilized in a reactive zone. Following binding of the sample analyte, the complex is reacted with labeled specific binding reagents (e.g., an enzyme-antibody conjugate) and analyte detected (e.g., upon reaction with substrate). In the case of a competition assay, analyte capture reagents are immobilized at the reactive zone and are contacted simultaneously with sample analyte and labeled analyte (e.g., an analyte-enzyme conjugate). The amount of label detected at the reactive zone is inversely proportional to the amount of analyte in the sample.

Another embodiment of the invention provides a device that is suitable for a lateral flow assay. For example, a test sample is added to a flow matrix at a first region. The test sample is carried by capillary action to a second region of the flow matrix where a particulate label capable of binding and forming a first complex with an analyte in the test sample. The particulate label can be a colored latex particle, dye sol, or gold sol conjugated to, for example, an antibody specific for a D. immitis antigen or polypeptides specific for B. burgdorferi or E. canis antibodies. The first complex is carried to a third region of the flow matrix where an antibody that specifically binds a D. immitis antigen is immobilized at a distinct location, a polypeptide that specifically binds an antibody specific for B. burgdorferi is immobilized at a distinct location, and a polypeptide that specifically binds an antibody specific for E. canis is immobilized at a distinct location. A second complex is formed between an immobilized antibody or a polypeptide and a first complex. For example, a first complex comprising a gold sol particle and antibody specific for a D. immitis antigen will specifically bind and form a second complex with an immobilized antibody specific for D. immitis. The particulate label that is part of the second complex can be directly visualized.

Any or all of the above embodiments can be provided as a kit. In one particular example, such a kit would include a device, e.g., as shown in FIG. 2, complete with specific binding reagents (e.g., a non-immobilized labeled specific binding reagent and an immobilized analyte capture reagent) and wash reagent, as well as detector reagent and positive and negative control reagents, if desired or appropriate. In addition, other additives can be included, such as stabilizers, buffers, and the like. The relative amounts of the various reagents can be varied, to provide for concentrations in solution of the reagents that substantially optimize the sensitivity of the assay. Particularly, the reagents can be provided as dry powders, usually lyophilized, which on dissolution will provide for a reagent solution having the appropriate concentrations for combining with a sample.

A device of the invention can also comprise an antibody or fragment thereof that specifically binds a D. immitis antigen immobilized on a solid support at a distinct location; a polypeptide that specifically binds an antibody specific for B. burgdorferi immobilized on the solid support at a distinct location; and a polypeptide that specifically binds an antibody specific for E. canis immobilized on the solid support at a distinct location. Detection of immunocomplexes on the solid support can be by any means known in the art.

Polypeptides of the Invention

Polypeptides that specifically bind an antibody or antibody fragment specific for E. canis, or B. burgdorferi can be immobilized analyte capture reagents of the invention. In this context “specifically binds” or “specific for” means that the polypeptide recognizes and binds to an anti-E. canis or anti-B. burgdorferi antibody, but does not substantially recognize and bind other molecules in a test sample. A polypeptide that specifically binds an antibody specific for E. canis can comprise any polypeptide that specifically binds an antibody specific for E. canis. In one embodiment of the invention a polypeptide specifically binds an antibody specific for E. canis wherein the antibody is produced by a mammal that is infected with E. canis. A polypeptide can be, for example, KSTVGVFGLKHDWDGSPILK (SEQ ID NO:2), which is derived from E. canis P30-1, or NTTTGVFGLKQDWDGATIKD (SEQ ID NO:3), which is derived from E. canis P30, or a combination thereof. A combination of polypeptides shown in SEQ ID NOs:2 and 3 can be, for example a 50/50 mixture of each polypeptide.

A polypeptide that specifically binds an antibody or antibody fragment specific for B. burgdorferi can comprise any polypeptide that specifically binds an antibody specific for B. burgdorferi. In one embodiment of the invention a polypeptide specifically binds an antibody specific for B. burgdorferi, wherein the antibody is produced by a mammal that is infected with B. burgdorferi. A polypeptide can be, for example, MKKDDQIAAAMVLRGMAKDGQFALK (SEQ ID NO:1) or MKKDDQIAAAMVLRGMAKDGQFALKD (SEQ ID NO:4). See e.g., WO 00/65064.

In one embodiment of the invention, the immobilized polypeptides are conjugated to bovine serum albumin (BSA). Polypeptides of the invention can either be full-length polypeptides or fragments of polypeptides. For example, fragments of polypeptides of the invention can comprise about 5, 8, 10, 15 or 20 amino acids of SEQ ID NOs:1-4. The invention also includes polypeptide variants that have substantial biological activity. That is, about 90% to about 110% of the biological activity of SEQ ID NOs:1-4. Such variants can include deletions, insertions, inversions, repeats, and substitutions selected according to general rules known in the art so as have little effect on activity. For example, guidance concerning how to make phenotypically silent amino acid substitutions is provided in Bowie et al., Science, 247:1306-1310 (1990). This reference describes two main strategies for studying the tolerance of an amino acid molecule to change.

The first strategy exploits the tolerance of amino acid substitutions by natural selection during the process of evolution. By comparing amino acid sequences in different species, the amino acid positions that have been conserved between species can be identified. These conserved amino acids are likely important for protein function. In contrast, the amino acid positions in which substitutions have been tolerated by natural selection indicate positions that are not critical for protein function. Thus, positions tolerating amino acid substitution can be modified while still maintaining biological activity of a polypeptide.

The second strategy uses genetic engineering to introduce amino acid changes at specific positions of a cloned gene to identify regions critical for protein function. For example, site-directed mutagenesis or alanine-scanning mutagenesis (the introduction of single alanine mutations at every residue in the molecule) can be used (Cunningham et al., Science, 244:1081-1085 (1989)). The resulting variant polypeptides can then be tested for biological activity by, for example immunohistochemical assay, an enzyme-linked immunosorbant assay (ELISA), a radioimmunoassay (RIA), or a western blot assay. Polypeptides of the invention can comprise at least 1, 2, 3, 4, 5, 7, or 10 conservative amino acid substitutions.

According to Bowie et al., these two strategies have revealed that proteins are surprisingly tolerant of amino acid substitutions. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

Besides conservative amino acid substitution, variant polypeptide molecules of the present invention include: (i) fusion of the mature polypeptide with another compound, such as a compound to increase the stability and/or solubility of the polypeptide (e.g., polyethylene glycol); (ii) fusion of the polypeptide with additional amino acids, such as an IgG Fc fusion region peptide, a leader or secretory sequence, or a sequence facilitating purification; (iii) synthesis of the polypeptide with additional amino acids that could, in turn, be used to conjugate the polypeptide to protein (e.g., bovine serum albumin) or assay reagent (e.g., horseradish peroxidase). Such variant polypeptides are deemed to be within the scope of those skilled in the art from the teachings herein.

Antibodies of the Invention

Antibodies or antibody fragments specific for D. immitis can be immobilized analyte capture reagents of the invention. Antibodies of the invention are antibody molecules that specifically and stably bind to a D. immitis antigen. An antibody or fragments thereof can be a polyclonal antibody, a monoclonal antibody, a single chain antibody (scFv), a chimeric antibody, or a fragment of an antibody. Fragments of antibodies are a portion of an intact antibody comprising the antigen binding site or variable region of an intact antibody, wherein the portion is free of the constant heavy chain domains of the Fe region of the intact antibody. Examples of antibody fragments include Fab, Fab′, Fab′-SH, F(ab′)₂ and Fᵥ fragments.

An antibody of the invention can be any antibody class, including for example, IgG, IgM, IgA, IgD and IgE. An antibody can be made in vivo in suitable laboratory animals or in vitro using recombinant DNA techniques. Means for preparing and characterizing antibodies are well know in the art. See, e.g., Dean, Methods Mol Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Immunol. 12:125-68(1992). For example, polyclonal antibodies can be produced by administering a polypeptide specific for D. immitis to an animal, such as a human or other primate, mouse, rat, rabbit, guinea pig, goat, pig, cow, sheep, donkey, chicken, or horse. For example, an antibody raised against the trichloroacetic acid (TCA) soluble fraction of disrupted heartworms can be administered to a chicken or rabbit. Serum or eggs from the immunized animal is collected and the antibodies are purified from the plasma or eggs by, for example, precipitation with ammonium sulfate, followed by chromatography, such as affinity chromatography. Techniques for producing and processing polyclonal antibodies are known in the art.

Additionally, monoclonal antibodies directed against a D. immitis antigen can also be readily produced. For example, normal B cells from a mammal, such as a mouse, which was immunized with a D. immitis antigen can be fused with, for example, HAT-sensitive mouse myeloma cells to produce hybridomas. Hybridomas producing D. immitis-specific antibodies can be identified using RIA or ELISA and isolated by cloning in semi-solid agar or by limiting dilution. Clones producing D. immitis-specific antibodies are isolated by another round of screening. Monoclonal antibodies can be screened for specificity using standard techniques, for example, by binding a D. immitis antigen to a microtiter plate and measuring binding of the monoclonal antibody by an ELISA assay. Techniques for producing and processing monoclonal antibodies are known in the art. See e.g., Kohler & Milstein, Nature, 256:495 (1975). Particular isotypes of a monoclonal antibody can be prepared directly, by selecting from the initial fusion, or prepared secondarily, from a parental hybridoma secreting a monoclonal antibody of a different isotype. Antibodies of the invention can also be chemically constructed. See, e.g., U.S. Pat. No. 4,676,980.

Immobilization of one or more analyte capture reagents onto a device or solid support is performed so that an analyte capture reagent will not be washed away by wash procedures, and so that its binding to analytes in a test sample is unimpeded by the solid support or device surface. One or more analyte capture reagents can be attached to a surface by physical adsorption (i.e., without the use of chemical linkers) or by chemical binding (i.e., with the use of chemical linkers). Chemical binding can generate stronger attachment of specific binding substances on a surface and provide defined orientation and conformation of the surface-bound molecules.

A polypeptide or antibody of the invention, i.e., an immobilized analyte capture reagent can be immobilized on a solid support or in a detection zone of a device of the invention. Immobilized analyte capture reagents can be immobilized at a distinct location of the support or device. A distinct location is a specific, known area of a substrate to which an analyte capture reagent is immobilized.

The methods of the invention detect Ehrlichia canis, Dirofilaria immitis, Borrelia burgdorferi antigens, antibodies, and/or antibody fragments in a test sample, such as a biological sample, an environmental sample, or a laboratory sample. A biological sample can include, for example, sera, blood, cells, plasma, or tissue from a mammal such as a dog, cat, or a human. The test sample can be untreated, precipitated, fractionated, separated, diluted, concentrated, or purified before application to a device of the invention.

Detection of analytes can be accomplished by, for example, ELISA, western blot, Immuno-fluorescent assay, radio-immuno assay, fluorescent polarization immunoassay and reversible flow chromatographic binding assay procedures.

All references cited in this disclosure are incorporated herein by reference.

We claim:

1. A device for detection of Dirofilaria immitis, Borrelia burgdorferi, and Ehrlichia canis antigens, antibodies, or fragments thereof comprising:
(a) an antibody that specifically binds a D. immitis antigen immobilized on a solid support at a distinct location;
(b) a polypeptide that specifically binds an antibody specific for B. burgdorferi immobilized on the solid support at a distinct location;
(c) a polypeptide that specifically binds an antibody specific for E. canis immobilized on the solid support of at a distinct location.

2. The device of claim 1, wherein the antibody that specifically binds to a D. immitis antigen is a polyclonal antibody.

3. The device of claim 2, wherein the antibody that specifically binds to a D. immitis antigen is a monoclonal antibody.

4. The device of claim 1, wherein the polypeptide that specifically binds an antibody specific for B. burgdorferi is derived from an invariable region of a variable domain of a variable surface antigen of B. burgdorferi (VlsE).

5. The device of claim 4, wherein the polypeptide that specifically binds an antibody specific for B. burgdorferi is selected from the group consisting of polypeptides shown in SEQ ID NO:1 and SEQ ID NO:4.

6. The device of claim 1, wherein the polypeptide that specifically binds an antibody specific for E. canis is a P30 or P30-1 polypeptide or a fragment thereof.

7. The device of claim 6, wherein the polypeptide that specifically binds an antibody specific for E. canis is selected from the group consisting of SEQ ID NO:2, SEQ ID NO:3 and a combination thereof.

8. A method of determining the presence or absence of D. immitis, B. burgdorferi, and E. canis antigens, antibodies, or fragments thereof in a biological sample comprising: applying the sample to the device of claim 1 and detecting formation or lack of formation of immunocomplexes on the device.

9. A device for the detection of Dirofilaria immitis, Borrelia burgdorferi, and Ehrlichia canis antigens, antibodies, or fragments thereof comprising:
(a) an elongated solid phase flow matrix comprising
(i) a first region for the receipt of a fluid sample
(ii) a second region wherein an antibody that specifically binds a D. immitis antigen is immobilized at a distinct location; a polypeptide that specifically binds an antibody specific for B. burgdorferi is immobilized at a distinct location, and a polypeptide that specifically binds an antibody specific for E. canis is immobilized at a distinct location;
(iii) a third region for application of a liquid detector reagent capable of removing unbound substances from the second region;
the second region being positioned intermediate to the first region and the third region;
(b) an absorbent reservoir of high volume capacity, wherein prior to use of the device, the absorbent reservoir is not in fluidic contact with the flow matrix; the device further comprising means for establishing fluidic contact between the absorbent reservoir and the flow matrix at positions selected so that the second region is between the absorbent reservoir and the third region;
(c) a sealed container of the liquid detector reagent positioned to be introduced at the third region of the matrix;
whereby the flow matrix and the regions thereof are sized and positioned to cause the fluid sample to flow initially along the elongated flow matrix in one direction toward and through the second region, and subsequently, upon introduction of the liquid detector reagent into the third region of the flow matrix, the liquid detector reagent to flow along the elongated flow matrix in a second direction opposite the first direction, through the second region, and into the absorbent reservoir, drawing unbound substances with it.

10. The device of claim 9, wherein the antibody that specifically binds to a D. immitis antigen is a polyclonal antibody.

11. The device of claim 10, wherein the antibody that specifically binds to a D. immitis antigen is a monoclonal antibody.

12. The device of claim 9, wherein the polypeptide that specifically binds an antibody specific for B. burgdorferi is derived from an invariable region of a variable domain of a variable surface antigen of B. burgdorferi (VlsE).

13. The device of claim 12, wherein the polypeptide that specifically binds an antibody specific for B. burgdorferi is selected from the group consisting of polypeptides shown in SEQ ID NO:1 and SEQ ID NO:4.

14. The device of claim 12, wherein the polypeptide that specifically binds an antibody specific for E. canis is a P30 and P30-1 polypeptide or fragment thereof.

15. The device of claim 14, wherein the polypeptide that specifically binds an antibody specific for E. canis is selected from the group consisting of SEQ ID NO:2, SEQ ID NO:3 and a combination thereof.

16. The device of claim 9, further comprising reagents that undergo a detectable reaction such that analyte bound at the second region is detected, and wherein the liquid reagent comprises a predetermined limited quantity of an inhibitor of the detectable reaction, wherein flow of the liquid reagent transports the inhibitor to the second region and then transports the inhibitor and unbound substances away from the second region, wherein the detectable reaction takes place in the absence of the unbound substances.

17. The device of claim 9, wherein, prior to use of the device, the absorbent reservoir is positioned so as not to contact the flow matrix, and wherein the device further comprises means for moving the absorbent reservoir into fluidic contact with the flow matrix.

18. The device of claim 9, further comprising a housing comprising:
(a) the elongated flow matrix;
(b) the sealed container of the liquid reagent; and
(c) means for applying the liquid reagent from the container to the third region of the flow matrix.

19. The device of claim 18, wherein the means for applying the liquid reagent to the flow matrix comprises a lance positioned and adapted to pierce the container.

20. The device of claim 9, comprising a housing containing:
(a) the elongated flow matrix;
(b) the sealed container of the liquid reagent; and
(c) means for applying the liquid reagent from the container to the third region of the flow matrix,
the means for moving the absorption reservoir into fluidic contact with the flow matrix being connected to the means for applying the liquid reagent, whereby an operator activates both the means in a single operation.

21. The device of claim 9, wherein the liquid reagent is a wash reagent and the flow matrix further comprises a fourth region for application of a detector reagent, wherein the third region is positioned intermediate to the second region and the fourth region.

22. The device of claim 21, the device comprising at least two sealed storage containers, one of the sealed storage containers containing a wash reagent, and one of the sealed storage containers containing the liquid detector reagent, both of the sealed containers being positioned proximal to the fourth region of the flow matrix.

23. The device of claim 22, wherein the device further comprises a means for applying the detector reagent and a means for applying the wash reagent, and the means for applying the detector reagent is connected to the means for applying the wash reagent, whereby an operator applies both the detector reagent and the wash reagent in a single operation.

24. The device of claim 23, wherein the means for applying the detector reagent and the wash reagent comprises a lance positioned and adapted to pierce both the containers.

25. The device of claim 9, further comprising at least one barrier comprising a soluble member positioned to block flow of the liquid reagent to the absorbent reservoir, whereby dissolution of the solid member permits fluid flow of the liquid reagent in the second direction to the absorbent reservoir after a predetermined time selected to be sufficient to permit sample to flow in the first direction through the second region.

26. The device of claim 25, wherein the barrier is positioned between the first region and the absorbent reservoir.

27. The device of claim 25, wherein the barrier is positioned between the third region and the second region.

28. The device of claim 25, comprising two of the barriers, one of the barriers being positioned between the first region and the absorbent reservoir, the second barrier being positioned between the third region and the second region.

29. A method for performing an assay that determines presence or absence of Dirofilaria immitis antigens, Borrelia burgdorferi antibodies, and Ehrlichia canis antibodies in a fluid sample by detecting binding of the antigens and antibodies to at least one immobilized antibody that specifically binds a D. immitis antigen, at least one immobilized polypeptide that specifically binds an antibody specific for B. burgdorferi, and at least one immobilized polypeptide that specifically binds an antibody specific for E. canis after washing unbound material from the immobilized antibody and polypeptides, the method comprising:
(a) providing
(i) an elongated solid phase flow matrix, the solid phase flow matrix capable of driving capillary fluid movement, the flow matrix comprising
(a) a first region for the receipt of a fluid sample;
(b) a second region at which the at least one antibody and at least one polypeptides are immobilized;
(c) a third region for application of a liquid detector reagent capable of removing unbound substances from the second region; the second region being positioned intermediate to the first region and the third region;
(ii) an absorbent reservoir of high volume capacity, wherein, prior to performing the method, the absorbent reservoir is not in fluidic contact with the flow matrix;
(b) applying the fluid sample to the first region of the flow matrix;
(c) allowing the fluid sample to flow in first direction through the second region, and then introducing the liquid detector reagent into the flow matrix at the third region;
(d) moving the absorbent reservoir into fluidic contact with the flow matrix, such that the sample and the liquid detector regent flow in a second direction, opposite to the first direction; and
(e) detecting the D. immitis antigens, B. burgdorferi antibodies, and E. canis antibodies bound at the second region.

30. The method of claim 29, wherein the D. immitis antigens, B. burgdorferi antibodies, and E. canis antibodies bound at the second region is detected by reagents that undergo a detectable reaction, and the liquid reagent comprises a predetermined limited quantity of an inhibitor of the detectable reaction, whereby flow of the liquid reagent transports the inhibitor initially to the second region, and when the inhibitor and unbound substances are transported away from the second region, the detectable reaction takes place in the absence of the unbound substances.

31. The method of claim 29, wherein, prior to use of the method, the absorbent reservoir is positioned so as not to contact the flow matrix, and after the fluid sample flows in a first direction through the second region, the absorbent reservoir is moved into fluidic contact with the flow matrix such that the sample and the liquid reagent flow in a second direction opposite to the first direction, through the second region, and into the absorbent reservoir, drawing unbound substances with it.

32. The method of claim 29, wherein the liquid reagent is contained in a sealed container and is applied to the third region of the flow matrix by piercing the container.

33. The method of claim 31, wherein the absorbent reservoir is brought into fluidic contact with the flow matrix and the liquid reagent is applied to the third region of the flow matrix by a single operator action.

34. The method of claim 29 or 30, wherein the liquid reagent is a wash reagent and the flow matrix further comprises a fourth region for the application of a detector reagent, the third region being positioned intermediate to the second region and the fourth region.

35. The method of claim 34, wherein the wash reagent and the detector reagent are each contained in separate sealed containers and are applied to the flow matrix by simultaneously piercing each of the containers with at least one lance.

36. The method of claim 29, the method further comprising providing at least one barrier comprising a soluble member positioned to block flow of the liquid reagent to the absorbent reservoir, whereby dissolution of the solid member permits fluid flow of the liquid reagent in the second direction to the absorbent reservoir after a predetermined time selected to be sufficient to permit sample to flow in the first direction through the second region.

37. The method of claim 36, wherein the barrier is positioned between the first region and the absorbent reservoir.

38. The method of claim 36, wherein the barrier is positioned between the third region and the second region.

39. The method of claim 36, comprising two of the barriers, one of the barriers being positioned between the first region and the absorbent reservoir, the second of the barriers being positioned between the third region and the second region.

40. A device for performing an assay that determines presence or absence of Dirofilaria immitis antigens, Borrelia burgdorferi antibodies, and Ehrlichia canis antibodies in a fluid sample by detecting binding of the antigens and antibodies to at least one immobilized antibody that specifically binds a D. immitis antigen, at least one immobilized polypeptide that specifically binds an antibody specific for B. burgdorferi, and at least one immobilized polypeptide that specifically binds an antibody specific for E. canis by detecting binding of the D. immitis antigens, B. burgdorferi antibodies, and E. canis antibodies to at least one immobilized antibody or polypeptide after washing unbound material from the immobilized antibody and polypeptides, the device comprising:
(a) an elongated fluid flow matrix comprising,
(1) a first segment for receiving a fluid sample,
(2) a second region at which the antibody and polypeptides are immobilized,
(3) a third region for application of a liquid detector reagent capable of removing unbound substances from the second region,
(b) an absorbent reservoir of high volume capacity,
(c) a sealed container of the liquid detector reagent positioned to be introduced at the third region of the flow matrix,
(d) at least one soluble barrier positioned to block flow of the liquid detector reagent from the container to the absorbent reservoir,
the second region being positioned intermediate to the third region and the absorbent reservoir,
the soluble barrier blocking flow of the liquid detector reagent to the absorbent reservoir until after the sample has flowed from the first region through the second region, at which point the barrier dissolves permitting the liquid detector reagent to flow.

41. The device of claim 40, wherein the barrier is positioned between the liquid reagent container and the second region.

42. The device of claim 40, wherein the barrier is positioned between the first region and the absorbent reservoir.

43. The device of claim 41, wherein the barrier is positioned between the first region and the absorbent reservoir.


SEQUENCE LISTING (4 sequences)

SEQ ID NO: 1 - protein, 25 aa, Borrelia burgdorferi
MKKDDQIAAAMVLRGMAKDGQFALK

SEQ ID NO: 2 - protein, 20 aa, Ehrlichia canis
KSTVGVFGLKHDWDGSPILK

SEQ ID NO: 3 - protein, 20 aa, Ehrlichia canis
NTTTGVFGLKQDWDGATIKD

SEQ ID NO: 4 - protein, 26 aa, Borrelia burgdorferi
MKKDDQIAAAMVLRGMAKDGQFALKD